(19) United States
(12) Patent Application Publication
(10) Pub. No.: US 2010/0210928 A1
(43) Pub. Date: Aug. 19, 2010

(54) PULSE OXIMETER WITH CHANGEABLE STRUCTURE

(76) Inventor: Chang-An Chou, Taipei (TW)

Correspondence Address: Chang-An Chou, 3F, No. 100, Sec. 3. Mingsheng E. Rd., Taipei, 105, TW

(21) Appl. No.: 12/304,768

(22) PCT Filed: Jun. 15, 2007
(86) PCT No.: PCT/CN2007/001881

§ 371 Date: Dec. 15, 2008

(30) Foreign Application Priority Data
Jun. 16, 2006 (CN) 200610092863.1

(51) Int. Cl. A61B 5/00

(52) U.S. Cl. 600/323; 600/344

(57) ABSTRACT

A pulse oximeter with changeable structure includes a first housing, having a light emitting element and a light receiving element mounted therein, and a second housing, electrically connected to the first housing, wherein the circuit needed by the pulse oximeter is distributed into the first and the second housings, and a corresponding pair of assembling mechanisms are further mounted on the first and the second housings, respectively, so that the first and the second housings are capable of being assembled together to form one single combination.

### FIELD OF THE INVENTION

The present invention is related to a pulse oximeter, and more particularly to a pulse oximeter with changeable structure.

### BACKGROUND OF THE INVENTION

Since blood oxygen concentration and pulse rate are important physiological indexes, many kinds of pulse oximeters are developed. For example, a sensor in clipping type or adhering type connected with a separated device, as disclosed in U.S. Pat. Nos. 5,279,295 and 5,249,576. Sequentially, for user's moving convenience, the sensor and the device are combined together, as disclosed in U.S. Pat. No. 5,490,523, in which the processor, display, sensor and power supply are all gathered in one housing to form an all-in-one pulse oximeter. A further more development is that the function of wireless transmission is included, as disclosed by U.S. Pat. No. 6,731,962 B1.

As can be observed, even after a long period of developing time, most of the different pulse oximeters, as described above, are coexisted and none of them becomes the dominant product, so that, obviously, there is no particular one can completely satisfy all the demands.

Therefore, the object of the present invention is to provide a novel pulse oximeter whose structure can be distributed into two parts as detection and be centralized to become one compact unit during carrying, so as to conform to different requirements simply and immediately.

### SUMMARY OF THE INVENTION

The present invention provides a pulse oximeter with changeable structure which includes a first housing, having a light emitting element and a light receiving element mounted therein, and a second housing, electrically connected to the first housing, wherein the circuit needed by the pulse oximeter is distributed into the first and the second housings, and a corresponding pair of assembling mechanisms are further mounted on the first and the second housings, respectively, so that the first and the second housings are capable of being assembled together to form one single combination.

Preferably, the first and the second housings can be electrically connected through a connecting wire, a soft PCB or a silicone-wrapped circuit, and the assembling mechanisms can be a pair of buckling mechanisms, a pair of adhering mechanisms or a pair of slid-and-track mechanisms.

Advantageously, the first housing is implemented to be able to attach to a tested location of the user, and further, the attachment is implemented by clipping, circling, or adhering and the tested location is the fingertip, the root portion of finger, the ear or the forehead.

Moreover, the pulse oximeter may further include a fixing element, which can be a band, connected to the second housing for fixing the second housing on the user as the second housing and the first housing are separated.

Furthermore, the pulse oximeter further includes an attaching element, which can be a hanging band or a winding band, for attaching the pulse oximeter to the user.

Besides, the circuit further includes a processing unit, a power unit, a wireless transmission module for wirelessly communicating with an external device, and a display unit.

In addition, the pulse oximeter is use to detect blood oxygen concentration and/or heartbeat.

### BRIEF DESCRIPTION OF THE DRAWINGS

A more detailed understanding of the invention may be had from the following description of a preferred embodiment, given by way of example, and to be understood in conjunction with the accompanying drawings, wherein:

FIG. 1A is a schematic view showing a pulse oximeter of a preferred embodiment according to the present invention;

FIG. 1B is a back view showing a pulse oximeter of a preferred embodiment according to the present invention;

FIGS. 1C-1D are lateral views showing a pulse oximeter of a preferred embodiment according to the present invention;

FIGS. 2A-2E are schematic views respectively showing a pulse oximeter being fixed on the user through a fixing element according to different embodiments of the present invention;

FIG. 3 is a schematic view showing one kind of electrical connection between two housings of a pulse oximeter according to a preferred embodiment the present invention;

FIGS. 4A-4D are schematic views respectively showing a pair of assembling mechanisms implemented on a pulse oximeter according to different referred embodiments of the present invention; and

FIGS. 5A-5B are schematic views showing a pulse oximeter being attached to the user through an attaching element according to different preferred embodiments of the present invention.

### DETAILED DESCRIPTION OF THE PREFERRED EMBODIMENTS

Since the technique on the pulse oximeter is known for non-invasively and continuously measuring the concentration of a material in a living body, especially in the blood vessel, by utilizing different light absorbing characteristics of plural wavelengths of lights, the pulse oximeter with changeable structure of the present invention also measures light absorbing difference, so that, as known, the physiological signals that can be calculated from the light absorbing difference, such as, the blood oxygen concentration, heart beat, and blood pressure etc., are all part of the present invention.

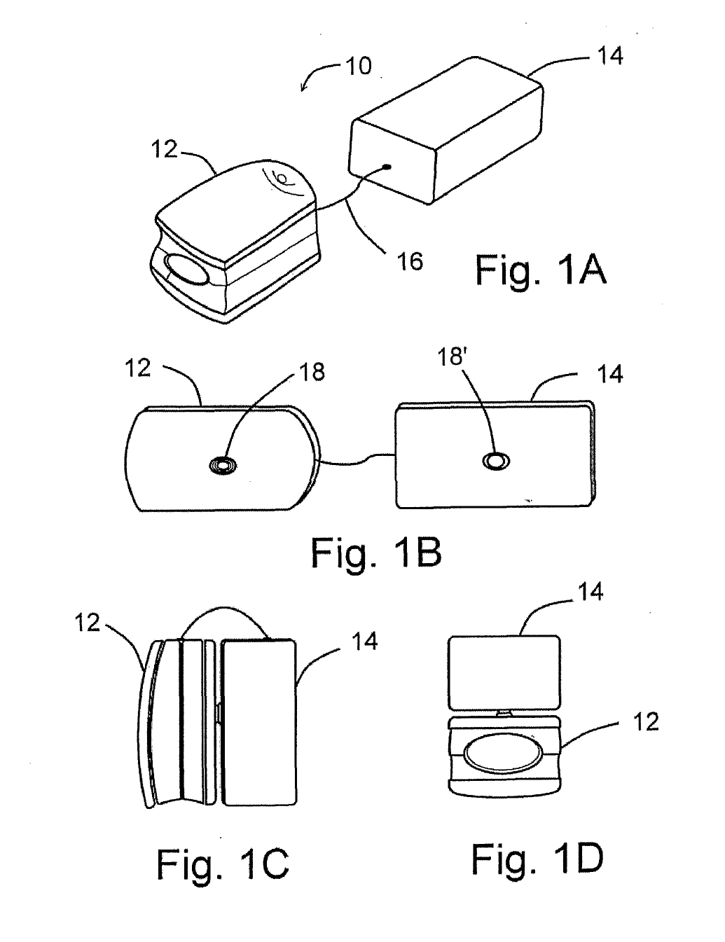

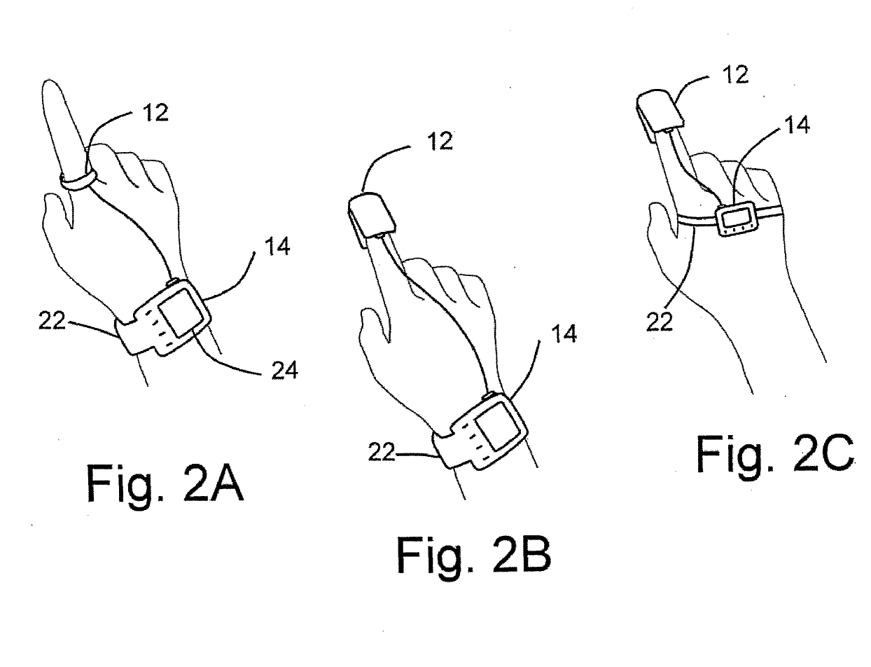

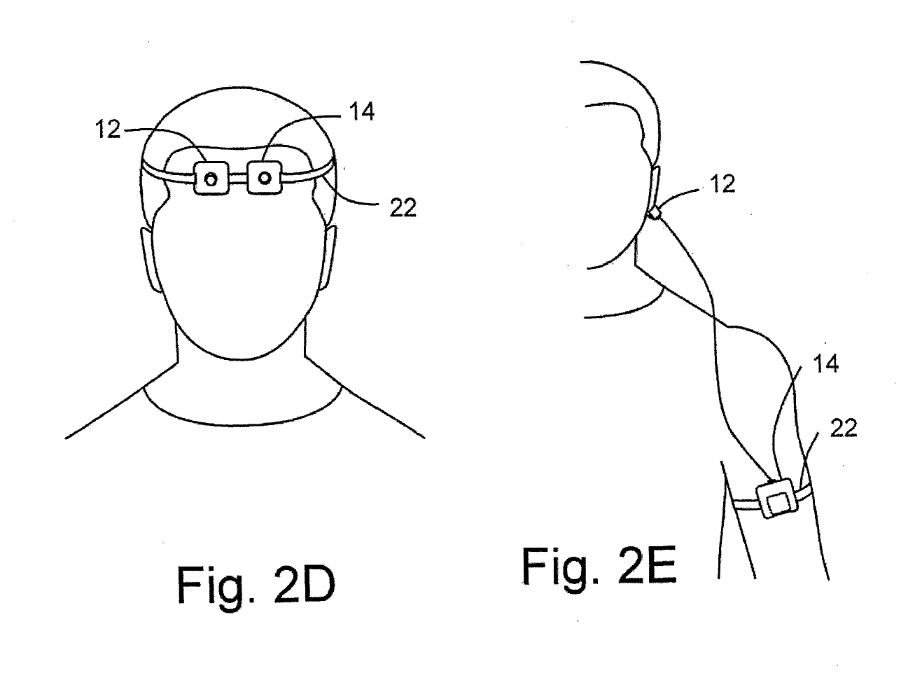

For achieving the object described above, the pulse oximeter with changeable structure according to the present invention has two states in structure: a combination state and a separation state. Please refer to FIG. 1, which is a schematic view showing the pulse oximeter with changeable structure according to the present invention. As shown, the pulse oximeter 10 has a first housing 12 and a second housing 14, which are electrically connected through a connecting wire 16. And, particularly, a corresponding pair of assembling mechanisms 18, 18′ are mounted on the two housings respectively. Here, the assembling mechanisms 18, 18′ are designed to be able to assemble together, so that the first housing 12 and the second housing 14 can be combined together through the assembling mechanisms, and then, one combined and compact unit, namely the combination state described above, is achieved. Accordingly, if there is the demand to separate the two housings, it only needs to disassemble the assembling mechanisms, and then the separation state can be recovered.

Under this concept, according to different demands, for example, different tested locations, different kinds of physiological signals and different attaching manners, the outer structures of the housings can be different. However, the invariable principle is one housing has to be implemented to contact the tested location of the user for processing the light detection. Therefore, in the present invention, the first housing is implemented to be the measuring component, so that a light emitting element and a light receiving element are included therein.

As to the second housing, it can be regarded as an extended component of the first housing because the circuit needed by the pulse oximeter are distributed into the first and the second housings, so that except the light emitting element and the light receiving element must be located in the first housing, other part of circuit needed by the pulse oximeter can be selected to locate in the first or the second housing according to the real demands, such as, volume, weight or appearance. Therefore, there is no other limitation to the circuit distribution.

Furthermore, in accordance with the present invention, after the assembling mechanisms are mutually assembled and connected together, the two housings are converted into one single object, which is equal to the conventional all-in-one pulse oximeter, so that in the combination state, the pulse oximeter of the present invention can own the advantages of the centralized pulse oximeter, for example, easy carried so as to respond to an emergency detection demand, or convenient stored. Besides, the pulse oximeter can further employ an attaching element, such as, a hanging band or a binding band, so that it can be more easily and stably carried by the user.

Moreover, when the assembling mechanisms are disassembled, the two housings are recovered to have the separation state. Now, the pulse oximeter is equal to the conventional ones, in which the sensor is connected with a device, and thus, the present invention can own the advantage thereof, for example, the loading at the tested location can be reduced, so that it will be more suitable for long-term monitoring, for instance, the blood oxygen concentration detection during sleep or in the hospital. The design of the second housing being able to separate from the first housing is equal to a weight reduction of pulse oximeter at the tested location.

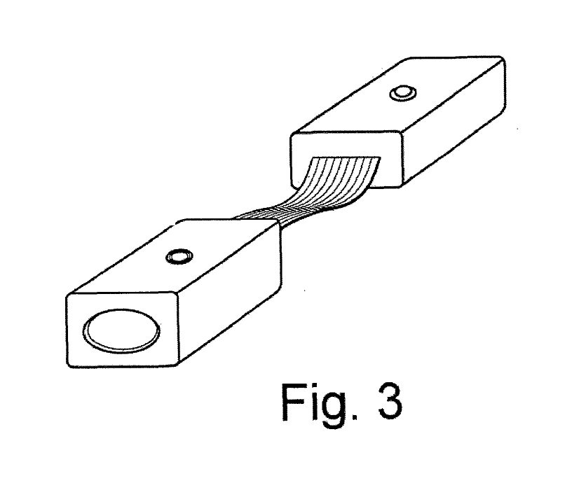

However, it should be noticed that, different from the conventional two-parts pulse oximeter, the second housing, after being separated form the first housing, will be mounted on the user, at a location other than the tested location, as shown in FIG. 2. For example, when the detection is carried out on the finger, the second housing can be arranged on the back of the hand or at the wrist (as shown in FIGS. 2A˜2C), or when the detection is proceeded on the head, the second housing can be arranged on the forehead (FIG. 2D) or the upper arm (FIG. 2E). Accordingly, the length of the connecting wire can be significantly reduced so as to replace the very long connecting wire employed by the conventional sensor for connecting to the far away processing device, and thus, the complexity can be reduced. More importantly, the user may therefore have a better mobility and will not be limited by the device aside the body. According to this design, for fixing the second housing on the user's body, the second housing can further have a fixing element 22, for example, for winding the forehead (FIG. 2D), the wrist (FIG. 2A and FIG. 2B), or the palm (FIG. 2C), and through this fixation, not only the second housing, the wiring between the housings will also be immobilized. Therefore, the pulse oximeter according to the present invention not only can distribute the volume and weight, but also, owing to the fixing element, can eliminate the wiring interference, which is common in the conventional technology, and achieve a more stable immobilization.

Consequently, as described above, the pulse oximeter according to the present invention not only can simultaneously own the advantages of two kinds of implementation types, but also can have a great mobility no matter in which condition.

Furthermore, a wireless transmission module can also be included in the present invention, so that, other than portability, the pulse oximeter according to the present invention also can achieve a wireless communication with an external device, for example, wirelessly communicating with a wireless transceiver of a personal computer, or wirelessly communicating with a portable device, such as a PDA or a notebook. And, because of the distributed structure, the increased volume caused from employing the wireless communication module can be easily distributed, so that the comfort during monitoring will not be scarified even a more convenient operation is achieved.

In addition to the wireless communication module, the pulse oximeter of the present invention also can include a display unit 24, no matter on the first housing or the second housing, for displaying real-time data or for guiding the user the operation, and further, the pulse oximeter also can include a power unit (not shown) for providing power, wherein a battery, chargeable or dischargeable, will be a better choice.

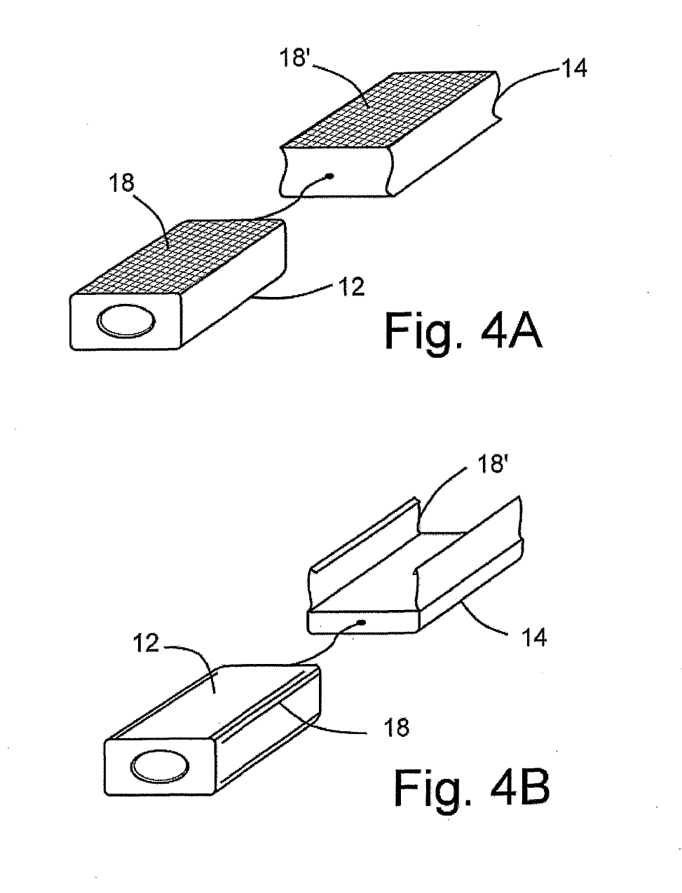

Besides, the connecting wire between the first and the second housings also can be changed corresponding to different situations. For example, the length thereof can be adjusted for matching to the distance between the first and the second housings, or an auto winding function can be employed for responding to different demands from different users. The material thereof also can have different choices, as shown in FIG. 3. For example, except the general connecting wire, soft PCB might be a great choice for simultaneously achieving connecting and wiring, or the electrical connecting wire wrapped by silicon also can achieve the identical purpose. When the material is more flexible, the fitting related to the body surface becomes better, so that the wiring interference can be reduced more. Therefore, according to the description, the connecting manner between the first and the second housings can have many different choices and not be limited.

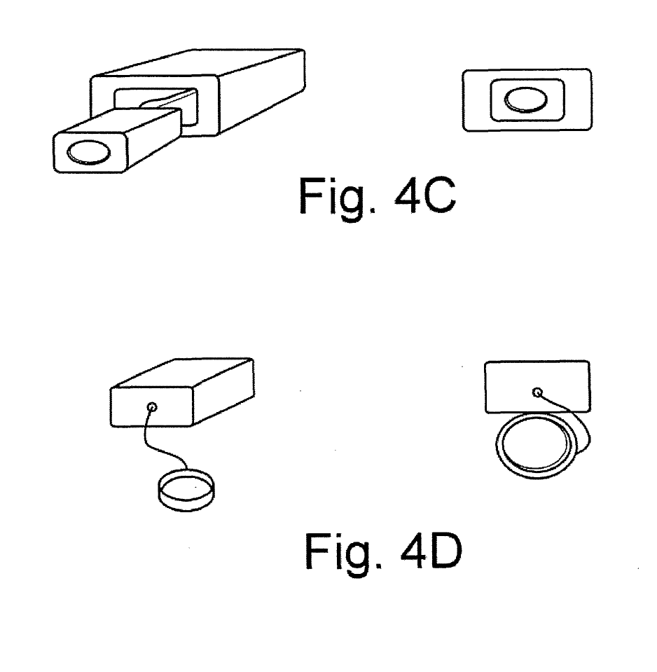

The assembling mechanisms 18, 18′ of the first and the second housings also can be implemented into different types. For example, except the buckling manner shown in FIG. 1, they also can be assembled by sliding track (FIG. 4B), adhering (FIG. 4A), combining (FIG. 4D) or sleeving (FIG. 4C) and only need to achieve the purpose of assembling.

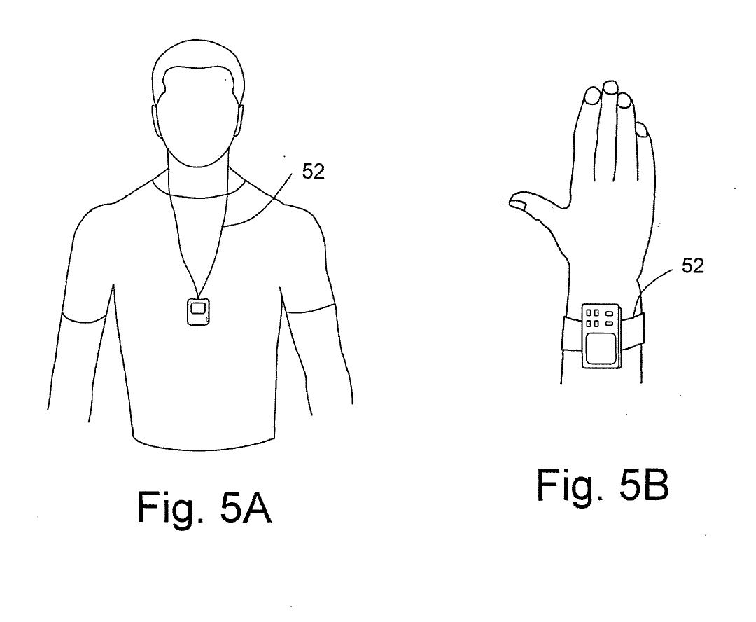

More advantageously, the structure of the pulse oximeter can be designed to conform to user's habit more. The outer structure of the first housing, which is used as the measuring part, can be modified according to different tested locations. For example, it can be implemented as the traditional ways of clipping and adhering at the fingertip, a ring sleeved on the finger, a patch on the forehead, or a clipper on the ear, as shown in FIG. 2. Besides, the second housing and the fixing element, which are regarded as the extended portion, even can have more variations. For example, they can be implemented as a watch when locating on the wrist, a part of a glove when locating on the back of hand, an arm-band when locating at the arm, and a head band when locating on the head, etc. In addition, as shown in FIG. 5, the attaching element 52 also has various choices. For example, the attaching element can be implemented as a hanging band, a wrist band, an arm band or a waist band, and preferably, when employing the wrist band, the attaching element and the fixing element can be implemented as one single watch band, as shown in FIGS. 2A˜2C, such that the pulse oximeter can be a normal watch if the measuring is not proceeded, and during the detection, the first and the second housings can be separated.

In the aforesaid, the pulse oximeter with changeable structure according to the present invention can easily be altered between the one-unit combination and the two separated parts through the distributable structure and the assembling mechanisms between the separated housings. By this structural change, the advantages of the conventional all-in-one pulse oximeter and the sensor with a processing device both can be achieved by the present invention, which is compact for portability and also separable for weight and volume distribution. Moreover, the attaching element and the fixing element respectively can provide the device the mobility and the fixation. In addition, if the wireless communication module is further included, then the pulse oximeter according to the present invention will have an even wider application range.

The above examples and disclosure are intended to be illustrative and not exhaustive. These examples and description will suggest many variations and alternatives to one of ordinary skill in this art. All these alternatives and variations are intended to be included within the scope of the attached claims. Those familiar with the art may recognize other equivalents to the specific embodiments described herein which equivalents are also intended to be encompassed by the claims attached hereto.

What is claimed is:

1. A pulse oximeter with changeable structure, comprising:
a first housing, having a light emitting element and a light receiving element mounted therein; and
a second housing, electrically connected to the first housing,
wherein
the circuit needed by the pulse oximeter is distributed into the first and the second housings; and
a corresponding pair of assembling mechanisms are further mounted on the first and the second housings, respectively, so that the first and the second housings are capable of being assembled together to form one single combination.

2. The pulse oximeter as claimed in claim 1, wherein the first and the second housings are electrically connected through a connecting wire, a soft PCB or a silicone-wrapped circuit.

3. The pulse oximeter as claimed in claim 1, wherein the first housing is implemented to be able to attach to a tested location of the user.

4. The pulse oximeter as claimed in claim 3, wherein the attachment is implemented by clipping, circling, or adhering.

5. The pulse oximeter as claimed in claim 3, wherein the tested location is the fingertip, the root portion of finger, the ear or the forehead.

6. The pulse oximeter as claimed in claim 1, wherein the assembling mechanisms are a pair of buckling mechanisms, a pair of adhering mechanisms or a pair of slid-and-track mechanisms.

7. The pulse oximeter as claimed in claim 1, further comprising a fixing element connected to the second housing for fixing the second housing on the user as the second housing and the first housing are separated.

8. The pulse oximeter as claimed in claim 7, wherein the fixing element is a band.

9. The pulse oximeter as claimed in claim 1, further comprising an attaching element for attaching the pulse oximeter to the user.

10. The pulse oximeter as claimed in claim 9, wherein the attaching element is a hanging band or a winding band.

11. The pulse oximeter as claimed in claim 1, wherein the circuit further comprises a processing unit and a power unit.

12. The pulse oximeter as claimed in claim 11, wherein the circuit further comprises a wireless transmission module for wirelessly communicating with an external device.

13. The pulse oximeter as claimed in claim 11, wherein the circuit further comprises a display unit.

14. The pulse oximeter as claimed in claim 1, wherein the pulse oximeter is use to detect blood oxygen concentration and/or heartbeat.
